(19) United States
(12) Patent Application Publication
(10) Pub. No.: US 2018/0015047 A1
(43) Pub. Date: Jan. 18, 2018

(54) MAGNETIC PARTICLE-BASED SUBSTANCE REMOVAL FROM TISSUE

(71) Applicant: Fortem Neurosciences, Inc., Los Angeles, CA (US)

(72) Inventors: Keith Black, Los Angeles, CA (US); Jack Kavanaugh, Los Angeles, CA (US)

(21) Appl. No.: 15/650,329

(22) Filed: Jul. 14, 2017

Related U.S. Application Data
(60) Provisional application No. 62/363,769, filed on Jul. 18, 2016.

(51) Int. Cl. A61K 9/50; A61M 25/01; A61K 9/00; A61B 17/32; A61B 17/00

(52) U.S. Cl. CPC A61K 9/5094; A61M 25/0127; A61K 9/0009; A61B 2017/00876; A61B 17/320092

(57) ABSTRACT

Provided herein are methods and compositions for removal of a substance using magnetic nanoparticles comprising a tagging element capable of targeting the nanoparticle to the substance. Further provided herein are methods and devices for removal of magnetic nanoparticle bound substances using a magnetic field generating device.

### CROSS-REFERENCE

This application claims priority to U.S. 62/363,769 filed Jul. 18, 2016, the entirety of which is incorporated by reference herein.

### BACKGROUND

Alzheimer's disease (AD) is the most common form of dementia. This incurable, degenerative, and terminal disease was first described by German psychiatrist and neuropathologist Alois Alzheimer in 1906 and was named after him. Generally, it is diagnosed in people over 65 years of age, although the less-prevalent early-onset Alzheimer's can occur much earlier. As of 2016, the number of diagnoses is reported to be 44 million-plus worldwide. AD is the sixth leading cause of death in the United States, where an estimated 1 in 9 people over the age of 65 is living with AD.

AD develops for an indeterminate period of time before becoming fully apparent, and it can progress undiagnosed for years. The mean life expectancy following diagnosis is approximately seven years. Fewer than three percent of individuals live more than fourteen years after diagnosis.

Currently used treatments offer a small symptomatic benefit; no treatments to delay or halt the progression of the disease are as yet available. As of 2014, more than 500 clinical trials have been conducted for identification of a possible treatment for AD, but it is unknown if any of the tested intervention strategies will show promising results.

What is needed are additional methods of treating central nervous system disorders such as Alzheimer's disease. Targeted treatment methods are particularly needed.

### SUMMARY

In certain embodiments, provided herein are compositions, devices, and methods to address the discussed need for targeted treatment in Alzheimer's disease. Such compositions, devices, and methods may also be useful for the treatment and/or diagnosis of other diseases or disorders.

In one aspect, provided herein is a method for removing a substance from a tissue, the method comprising: (a) providing the tissue comprising the substance, a magnetic coupling nanoparticle comprising a tagging element and a magnetic element, wherein the tagging element is capable of binding to the substance, and a magnetic field generating removal device configured to magnetically couple with the magnetic element of the nanoparticle; and (b) drawing the substance from the tissue using the magnetic field generating removal device when the substance is bound to the nanoparticle, thereby removing the substance from the tissue. In some embodiments, the tissue is brain tissue. In some embodiments, the substance comprises a misfolded protein, prion, microorganism, viral particle, pathogen, infectious agent, toxic agent, organic molecule, element, mercury, lead, cancer cells, inflammatory cells, or an associated plurality of the same. In some embodiments, the tagging element comprises an antibody or peptide that binds to a Tau protein or amyloid beta. In some embodiments, the magnetic field generating removal device generates a magnetic field that is capable of being turned on and off In some embodiments, the substance-bound nanoparticle magnetically couples with the magnetic field generating removal device when the magnetic field is on, thereby accumulating the substance. In some embodiments, the method further comprises releasing the substance-bound nanoparticle from the magnetic field generating removal device when the magnetic field is off. In some embodiments, the magnetic field generating removal device comprises a conduit configured to comprise a first configuration where the conduit is closed, and a second configuration where the conduit is open such that the substance-bound nanoparticle may enter the conduit. In some embodiments, the magnetic coupling nanoparticle is administered to an individual, and the magnetic field removal device is positioned within proximity to the tissue to draw the substance-bound nanoparticle from the individual using the magnetic field generating removal device.

In another aspect, provided herein is a magnetic field generating device comprising: a conduit configured to magnetically couple with and receive a magnetic element when in proximity to the magnetic element within a tissue. In some embodiments, generation of the magnetic field can be turned on and off. In some embodiments, the device comprises a shunt or catheter having a distal end from which the magnetic field is emitted. In some embodiments, the conduit is configured to comprise a closed configuration and an open configuration, wherein the magnetic field is configured to be off when the conduit is open and the magnetic field is configured to be on when the conduit is closed. In some embodiments, the device further comprises a valve.

In another aspect, provided herein is a system comprising: a nanoparticle comprising a tagging element configured to bind the nanoparticle to a substance; and a magnetic element configured to magnetically couple the nanoparticle to a magnetic field; and a removal device configured to generate the magnetic field. In some embodiments, the magnetic element comprises one or more of ferrous oxide, ferric oxide, ferromagnetic iron oxide, maghemite, gamma-Fe2O3, magnetite, Fe3O4. In some embodiments, the substance is a misfolded protein, prion, microorganism, viral particle, pathogen, infectious agent, toxic agent, organic molecule, element, mercury, lead, cancer cells, inflammatory cells, or an associated plurality of the same. In some embodiments, the removal device comprises a shunt, catheter, implant, or intermittent device. In some embodiments, generation of the magnetic field is adjustable, and/or the magnetic field can be turned on and off. In some embodiments, the device comprises a conduit configured to comprise an open configuration and a closed configuration.

In another aspect, provided herein is a method for removing a substance from a tissue comprising: (a) providing a magnetic coupling nanoparticle comprising a tagging element and a magnetic element, wherein the tagging element is capable of targeting the nanoparticle to the substance so that when the nanoparticle is located in the tissue, the nanoparticle specifically binds to the substance, and wherein the magnetic element is capable of magnetic coupling; and (b) providing a magnetic field generating removal device configured to magnetically couple with the substance and draw the substance from the tissue when the substance is bound to the nanoparticle, thereby removing the substance from the tissue. In some embodiments, the tissue comprises brain tissue. In some embodiments, the substance comprises a Tau protein or amyloid beta. In some embodiments, the substance comprises a misfolded protein, prion, microorganism, viral particle, pathogen, infectious agent, toxic agent, organic molecule, element, mercury, lead, cancer cells, inflammatory cells, or an associated plurality of the same. In some embodiments, the tagging element comprises an antibody or peptide. In some embodiments, the antibody or peptide is selected for binding affinity to the substance. In some embodiments, the substance is Tau protein. In some embodiments, the substance is amyloid beta. In some embodiments, the substance comprises a misfolded protein, prion, microorganism, viral particle, pathogen, infectious agent, toxic agent, organic molecule, element, mercury, lead, cancer cells, inflammatory cells, or an associated plurality of the same. In some embodiments, the antibody selectively binds Tau or amyloid beta. In some embodiments, the antibody is monoclonal or polyclonal. In some embodiments, the antibody is generated against or selected for binding affinity to a polypeptide comprising SEQ ID NO: 1 or a fragment or derivative thereof. In some embodiments, the tagging element comprises curcumin. In some embodiments, the magnetic element comprises ferrous, ferric oxide, ferromagnetic iron oxide, maghemite, gamma-Fe2O3, magnetite, or Fe3O4. In some embodiments, the removal device comprises a shunt or catheter. In some embodiments, generation of the magnetic field is adjustable. In some embodiments, generation of the magnetic field can be turned on and off In some embodiments, the substance bound to the nanoparticle magnetically couples with the removal device when the magnetic field is on, thereby accumulating the substance. In some embodiments, the substance bound to the nanoparticle releases from the removal device when the magnetic field is off, thereby allowing removal of the substance. In some embodiments, the method further comprises providing a conduit configured to comprise a first configuration and a second configuration. In some embodiments, the conduit comprises a distal end from which the magnetic field is emitted. In some embodiments, the first configuration, the distal end of the conduit is closed, such that the substance cannot enter the conduit. In some embodiments, the magnetic field is on, such that the substance magnetically couples with the distal end of the conduit. In some embodiments, the second configuration, the distal end of the conduit is open, such that the substance can enter the conduit. In some embodiments, the magnetic field is off, such that the substance is not magnetically coupled with the distal end of the conduit. In some embodiments, the method further comprises magnetically coupling the substance to the distal end of the conduit when the conduit is in the first configuration, and releasing the substance from the distal end of the conduit when the conduit is in the second configuration, such that the substance is received into the conduit, thereby removing the substance from the tissue. In some embodiments, the device further comprises an adjustable valve configured to control a pressure gradient between the conduit and the tissue, thereby controlling a flow rate into the conduit. In some embodiments, the valve is adjusted to favor the flow rate into the conduit when the conduit is in the second configuration. In some embodiments, the removal device further comprises a second shunt or catheter.

In another aspect, provided is a method comprising: (a) administering a magnetic coupling nanoparticle to an individual, wherein the nanoparticle comprises: (i) a tagging element configured to specifically bind the nanoparticle to a substance in a tissue of the individual; and (ii) a magnetic element configured to magnetically couple the substance to a magnetic field when the substance is bound to the nanoparticle; (b) positioning a removal device within proximity to the tissue, wherein the removal device is configured to generate the magnetic field; and (c) coupling the substance and the magnetic field so that the substance is drawn from the tissue by the removal device. In some embodiments, the removal device is configured to modulate the magnetic field. In some embodiments, the removal device comprises a conduit configured to receive the substance as it is removed from the tissue. In some embodiments, the removal device comprises an implanted device. In some embodiments, the removal device comprises a shunt or catheter. In some embodiments, the removal device further comprises a second shunt or catheter.

In another aspect, provided is a magnetic coupling nanoparticle comprising: a tagging element for binding the nanoparticle to a substance; and a magnetic element for coupling the nanoparticle to a magnetic force. In some embodiments, the nanoparticle further comprises one or more of ferrous, ferric oxide, ferromagnetic iron oxide, maghemite, gamma-Fe2O3, magnetite, or Fe3O4. In some embodiments, the tagging element comprises a peptide or antibody. In some embodiments, the nanoparticle further comprises a targeting ligand. In some embodiments, the targeting ligand mediates receptor-mediated transport. In some embodiments, the targeting ligand comprises transferrin. In some embodiments, the targeting ligand comprises insulin. In some embodiments, the targeting ligand is capable of bind a transferrin receptor. In some embodiments, the targeting ligand is capable of bind an insulin receptor. In some embodiments, the targeting ligand is capable of bind a low density lipoprotein receptor.

In another aspect, provided is a magnetic field generating device comprising: a conduit configured to magnetically couple with and receive a magnetic element when in proximity to the magnetic element within a tissue. In some embodiments, generation of the magnetic field is adjustable. In some embodiments, generation of the magnetic field can be turned on and off. In some embodiments, the device is a shunt or catheter. In some embodiments, the conduit comprises a distal end. In some embodiments, the magnetic field is emitted from the distal end. In some embodiments, the conduit is configured to comprise a first configuration and a second configuration. In some embodiments, the first configuration, the conduit is closed. In some embodiments, the magnetic field is on when the conduit is closed. In some embodiments, when in the second configuration, the conduit is open. In some embodiments, the magnetic field is off when the conduit is open. In some embodiments, the magnetic field is adjusted when the conduit is open. In some embodiments, the device further comprises a valve. In some embodiments, the valve is configured to control a pressure gradient, thereby controlling a flow rate into the conduit. In some embodiments, the device further comprises a second shunt or catheter.

In another aspect, provided is a system comprising: (a) a nanoparticle comprising (i) a tagging element configured to specifically bind the nanoparticle to a substance; and (ii) a magnetic element configured to magnetically couple the substance to a magnetic field when the tagging element is bound to the substance; and (b) a removal device configured to generate the magnetic field, wherein the removal device is configured to generate a magnetic field and thus remove the substance from a tissue of an individual when the substance is bound to the nanoparticle and the removal device is positioned within proximity to the tissue. In some embodiments, the magnetic element comprises one or more of ferrous oxide, ferric oxide, ferromagnetic iron oxide, maghemite, gamma-Fe2O3, magnetite, Fe3O4. In some embodiments, the tagging agent comprises an amino acid residue. In some embodiments, the tagging agent comprises an antibody or peptide. In some embodiments, the antibody or peptide is generated against or selected for binding affinity to SEQ ID NO: 1 or a fragment or derivative thereof. In some embodiments, the antibody or peptide is generated against or selected for binding affinity to a peptide comprising at least 3, 4, 5, 6, 7, or 8 contiguous amino acids of SEQ ID NO: 1. In some embodiments, the tagging agent comprises curcumin. In some embodiments, the substance is a misfolded protein or prion. In some embodiments, the misfolded protein is Tau or amyloid beta. In some embodiments, the substance comprises a microorganism. In some embodiments, the substance comprises a viral particle. In some embodiments, the substance comprises pathogen or infectious agent. In some embodiments, the substance comprises a cancer cell or inflammatory cell. In some embodiments, the substance is an organic molecule or element or an associated plurality of the same. In some embodiments, the substance is mercury or lead. In some embodiments, the substance comprises a lipid. In some embodiments, the substance comprises a bile component. In some embodiments, the substance comprises calcium. In some embodiments, the tissue comprises cerebrospinal fluid. In some embodiments, the tissue comprises blood. In some embodiments, the tissue comprises a kidney tissue. In some embodiments, the tissue comprises a brain tissue. In some embodiments, the tissue comprises a liver tissue. In some embodiments, the tissue comprises a lung tissue. In some embodiments, the removal device comprises a shunt, catheter, implant, or intermittent device. In some embodiments, generation of the magnetic field is adjustable. In some embodiments, generation of the magnetic field can be turned on and off In some embodiments, the device comprises a conduit. In some embodiments, the conduit comprises a distal end. In some embodiments, the magnetic field is emitted from the distal end. In some embodiments, the conduit is configured to comprise a first configuration and a second configuration. In some embodiments, when in the first configuration, the conduit is closed. In some embodiments, the magnetic field is on when the conduit is closed. In some embodiments, when in the second configuration, the conduit is open. In some embodiments, the magnetic field is off when the conduit is open. In some embodiments, the magnetic field is adjusted when the conduit is open. In some embodiments, the system further comprises a valve. In some embodiments, the valve is configure to control a pressure gradient, thereby controlling a flow rate into the conduit. In some embodiments, the system further comprises a second shunt or catheter.

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

FIG. 1 depicts an embodiment of a magnetic field generating device as described herein.

FIG. 2 depicts an embodiment of a nanoparticle and a device as disclosed herein, said nanoparticle comprising a first portion comprising a tagging element configured to bind to a substance of interest.

FIG. 3 depicts an embodiment of a method for removing a substance using a nanoparticle and a device as disclosed herein, said device being depicted in a first configuration.

FIG. 4 depicts an embodiment of a method for removing a substance using a nanoparticle and a device as disclosed herein, said device being depicted in a second configuration.

### DETAILED DESCRIPTION

Described herein are compositions, systems, devices, and methods for the identification and/or removal of a substance of interest from a mammalian tissue. The compositions described herein comprise nanoparticles that are configured to selectively bind to a substance of interest that is present in a mammalian tissue, and then facilitate one or more of: a) the identification of the presence of the substance in the tissue and b) the removal of the substance from the tissue. A nanoparticle as described herein comprises a first portion configured to selectively bind the nanoparticle to a substance and a second portion configured to facilitate that identification and/or removal of the substance from the tissue.

In some embodiments, a first portion of a nanoparticle as described herein is configured to bind to a substance of interest in a mammalian tissue. For example, in some embodiments, the first portion of the nanoparticles is configured to selectively bind to a substance in mammalian brain tissue. For example, the first portions of the nanoparticles, in some embodiments, are configured to bind to a substance comprising Tau and/or amyloid beta protein in the tissue of a mammalian brain. For example, in some embodiments, the first portion of the nanoparticle comprises an anti-Tau and/or anti-amyloid beta antibody domain. In these embodiments, the anti-Tau and/or anti-amyloid beta protein antibody domains of a nanoparticle selectively bind a Tau and/or amyloid beta protein in a tissue of a mammalian brain and thus form a complex comprising the nanoparticle bound to one or more Tau and/or amyloid beta proteins. That is, in some embodiments, one nanoparticle selectively binds to one molecule in the tissue of a mammal and in some embodiments, one nanoparticle selectively binds to a plurality of substances. For example, in some embodiments, one nanoparticle selectively binds to a Tau protein forming a complex comprising the nanoparticle and the Tau protein. For example, in some embodiments one nanoparticle selectively binds to two Tau proteins (e.g., where the nanoparticle comprises two or more anti-Tau antibody domains) forming a complex comprising one nanoparticle and two Tau proteins. For example, in some embodiments, a plurality of nanoparticles selectively bind to a single Tau protein forming a complex comprising a plurality of nanoparticles and the Tau protein. For example, in some embodiments, one nanoparticle selectively binds to an amyloid beta protein forming a complex comprising the nanoparticle and the amyloid beta protein. For example, in some embodiments one nanoparticle selectively binds to two amyloid beta proteins (e.g., where the nanoparticle comprises two or more anti-amyloid beta antibody domains) forming a complex comprising one nanoparticle and two amyloid beta proteins. For example, in some embodiments, a plurality of nanoparticles selectively bind to a single amyloid beta protein forming a complex comprising a plurality of nanoparticles and the amyloid beta protein. For example, in some embodiments, one nanoparticle selectively binds to a Tau protein and an amyloid beta protein (e.g., where the nanoparticle comprises both anti-Tau and an anti-amyloid beta antibody domains) forming a complex comprising the nanoparticle, the Tau protein, and the amyloid beta protein.

In some embodiments, a second portion of the nanoparticles described herein is configured to couple the nanoparticle to a device, for example, a diagnostic and/or therapeutic device. For example, in some embodiments a second portion of the nanoparticles described herein comprises a substance capable of forming a magnetic coupling, for example, a metal and/or magnet. A nanoparticle selectively bound to a substance in a tissue of a mammal forming a complex as described herein may couple with a magnetic field generated by, for example, a diagnostic or therapeutic device. In some embodiments, a complex as described herein is imaged by a Magnetic Resonance Imaging (MRI) device through a magnetic coupling formed between the magnetic field generated by the MRI device and one or more second portions of one or more nanoparticles in a complex with a one or more substances. In these embodiments, a substance that would otherwise not be visible on imaging is visualized using MRI when bound to the nanoparticles as described herein. In some embodiments, a device generates a magnetic field in proximity to a tissue containing a complex as described herein and thus draws the complex (via the second portion of one or more nanoparticles) out of the tissue and towards the device. For example, in some embodiments, a device implanted into the body of a mammal generates a magnetic field that is configured to draw the nanoparticle selectively bound to a substance in a tissue out of the tissue and eventually out of the body of the mammal. For example, in some embodiments, a device comprises a cerebral shunt configured to generate a magnetic field that couples to a second portion of a nanoparticle selectively bound to a Tau and/or amyloid beta protein in a brain tissue. In these embodiments, the generated magnetic field draws the complex comprising the nanoparticle and the Tau and/or amyloid beta protein out of the brain tissue and into the Cerebral Spinal Fluid (CSF) through a coupling of the magnetic field to the second portion of the nanoparticle which, for example, comprises a metal or magnet. In these embodiments, the generated magnetic field that draws the complex out of the brain tissue and into the CSF ultimately facilitates removal of the complex from the body when the CSF containing the complex is removed from the body. Removal of substances from tissue as described herein provides, for example, both therapeutic (i.e. removal of toxic substances) and diagnostic (i.e. removal of substances for identification purposes) benefits.

The present disclosure also relates to magnetic nanoparticles which provide dual function as diagnostic and therapeutic agents. In particular, in one aspect, the present disclosure relates to compositions comprising magnetic nanoparticles and their use as targeted therapeutic agents for Alzheimer's disease and related diseases and conditions.

Some embodiments of the present disclosure provide systems, methods, and devices for diagnosing and treating any number of diseases, such as diseases spatially localized in or around tissue and organs. Some embodiments of the present disclosure provide the advantage of allowing concurrent targeted diagnosis and treatment using the same magnetic nanoparticles, for example by magnetically coupling the nanoparticles to follow targeting to a magnetic field generating device. Some embodiments relate to removal of a substance from an environment, such as tissue, using a magnetic nanoparticle capable of targeting the substance, and a magnetic field generating device capable of magnetically coupling with the magnetic nanoparticle.

In some embodiments, the present invention provides a system comprising a nanoparticle comprising a first portion configured to bind a substance of interest, such as a tagging element, and a second portion configured to couple the nanoparticle to a device, such as by magnetic coupling. For example, provided herein are systems comprising a) a magnetic nanoparticle comprising a tagging element, wherein the tagging element targets the nanoparticle to a substance of interest; and b) a magnetic field generating device, wherein the magnetic nanoparticle magnetically couples with the device when a magnetic field is emitted. In some embodiments, the tagging element is an antibody or peptide. Non-limiting examples of substances of interest include an amyloid beta protein, oligomer, and fibril. In some embodiments, the system further comprises an imaging device, such as an Mill device. In some embodiments, nanoparticles are administered and/or targeted via injection or other delivery method to the substance of interest, for example, at a disease site.

Some embodiments of the present disclosure provide a method comprising administering to a subject a nanoparticle comprising a first portion configured to bind a substance of interest, such as a tagging element, and a second portion configured to couple the nanoparticle to a device, such as by magnetic coupling. In some examples, such a method comprises a) administering a magnetic nanoparticle comprising a tagging element, wherein the tagging element targets the nanoparticle to a substance of interest; b) detecting the presence of the substance of interest in the subject by identifying the magnetic nanoparticle; and optionally c) removing the substance of interest by magnetically coupling the nanoparticle to a magnetic field generating device, such as a shunt or catheter.

Some embodiments provide a method of treating Alzheimer's disease, comprising administering a nanoparticle comprising a first portion configured to bind a substance of interest, such as a tagging element, and a second portion configured to couple the nanoparticle to a device, such as by magnetic coupling. In some examples, such a method comprises: a) administering a magnetic nanoparticle comprising a tagging element, wherein the tagging element targets the nanoparticle to a substance of interest, for example an Alzheimer's disease specific molecule such as Tau and/or amyloid beta; and b) removing the nanoparticle by magnetically coupling the nanoparticle with a magnetic field generating device, such as a shunt or catheter. In some embodiments, magnetic nanoparticles are locally delivered directly at a disease site or through a catheter or similar delivery vehicle for subsequent monitoring, diagnosis, and/or therapy.

Some embodiments of the present disclosure provide a magnetic nanoparticle comprising a first portion configured to bind a substance of interest, such as a tagging element, and a second portion configured to couple the nanoparticle to a device, such as by magnetic coupling. In some examples, the first portion comprises a tagging element, such as an antibody or peptide. In some examples, the tagging element is selected for binding to a substance of interest, such as an Alzheimer's disease specific molecule, for example Tau and/or amyloid beta. The magnetic nanoparticle, in some embodiments, further comprises a targeting agent, said targeting agent capable of targeting the nanoparticle to a substance site, such as across the blood brain barrier.

Some embodiments of the present disclosure provide a magnetic field generating device capable of magnetically coupling with a portion of a magnetic nanoparticle. The device can comprise a conduit with a distal end opening which is capable of being in an open or closed configuration. The magnetic field generated by the device, in some embodiments, is adjustable (e.g., in terms of strength and/or orientation) and, in some embodiments, is configured to be turned on and turned off either automatically and/or by a user. In some embodiments, the device further comprises a valve configured to control and/or modulate the pressure within the conduit, thereby controlling the flow of fluid or particles into the conduit from the surrounding environment. In some embodiments, the device is a medical device such as a shunt, dual-shunt, or catheter.

### Alzheimer's Disease (AD)

Pathogenic events that initiate memory loss in AD are induced by the accumulation of potent neurotoxins. These neurotoxins arise from physiological proteins that mis-fold or mis-assemble, forming conformationally unique amyloid beta species (Koo et al., (1999) Proc. Natl. Acad. Sci. U.S. A 96, 9989-9990; Selkoe, (2004) Nat. Cell Biol 6, 1054-1061; Walsh and Selkoe, (2004) Protein Pept. Lett. 11, 213-228). Early memory loss is considered the consequence of synapse failure, not neuron death, and is now widely attributed to pathogenic amyloid beta oligomers instead of the fibrillar amyloid beta of amyloid plaques (Hardy & Selkoe, (2002) Science 297, 353-356; Rodgers et al., (2005). Progress report on Alzheimer's disease 2004-2005. November 2005. U.S. Department of Health and Human Services; National Institutes on Aging; National Institutes of Health; Klein et al., (2001) Trends Neurosci. 24, 219-224; Selkoe, (2008) Behay. Brain Res. 192, 106-113; Glabe, (2008) J. Biol. Chem). The presence of a pathological species demonstrably absent from healthy individuals provides a target for immunotherapy. In some embodiments, elimination of these toxins stops disease progression and/or reverses the dysfunction in cognitive impairment and AD. Oligomers have been detected in vitro and in brain since the early 1990's but only after 1998 (Lambert et al, (1998) Proc. Natl. Acad. Sci. U.S.A 95, 6448-6453) have they been recognized as putative neurotoxins responsible for dementia. Oligomers are extracellular ligands (Gong et al., Proc. Natl. Acad. Sci. U.S.A 100, 10417-10422) that bind to specific synapses (Lacor et al., (2004) J Neurosci. 24, 10191-10200). They are markedly increased in human AD patients and show perineuronal localization in AD human brain tissue (Gong et al., supra; Lacor et al., supra; Chang et al., (2003) J. Mol. Neurosci. 20, 305-313; Lambert et al., (2009) CNS. Neurol. Disord. Drug Targets. 8, 65-81; Lambert et al., (2007) J Neurochem. 100, 23-35). To study these toxic Amyloid beta species, antibodies have been developed that prevent binding of aggregated Ab and the resulting responses in cultured cells.

The properties of Amyloid beta oligomers and their role in Alzheimer's disease (AD) have become increasingly clear during the past decade (Viola et al, J Nutr Health 2008). Oligomers, unlike current drug targets, act as initiators of disease mechanisms and provide an optimal target for disease-modifying AD therapeutics. One approach with significant emerging interest is the use of nanotechnology for improved, more targeted, and less invasive diagnostics and therapeutics. An appealing feature of nanoparticles with enhanced magnetic or optical properties is their application for integrated diagnostic therapy. Functional conjugates of highly specific anti-Amyloid beta antibodies and magnetic nanoparticles are disclosed herein. In some embodiments, magnetic nanoparticles disclosed herein specifically target Amyloid beta oligomers and facilitate removal through magnetic coupling with a magnetic field generating device, such as a shunt.

Amyloid aggregate formation is seen at the beginning of a neurodegenterative cascade that eventually leads to neurotoxicity, oxidative stress, and neuroinflammation. The amyloid core of these plaques consists of fibrils composed of amyloid beta peptide variants and surrounded by dead neurons. Without being bound by theory, amyloid beta peptide length varies from 39-43 amino acids, with the most abundant forms being 40 and 42 amino acids in length. These amyloid beta oligomers can also be referred to as amyloid beta derived diffusible ligands, or ADDLs.

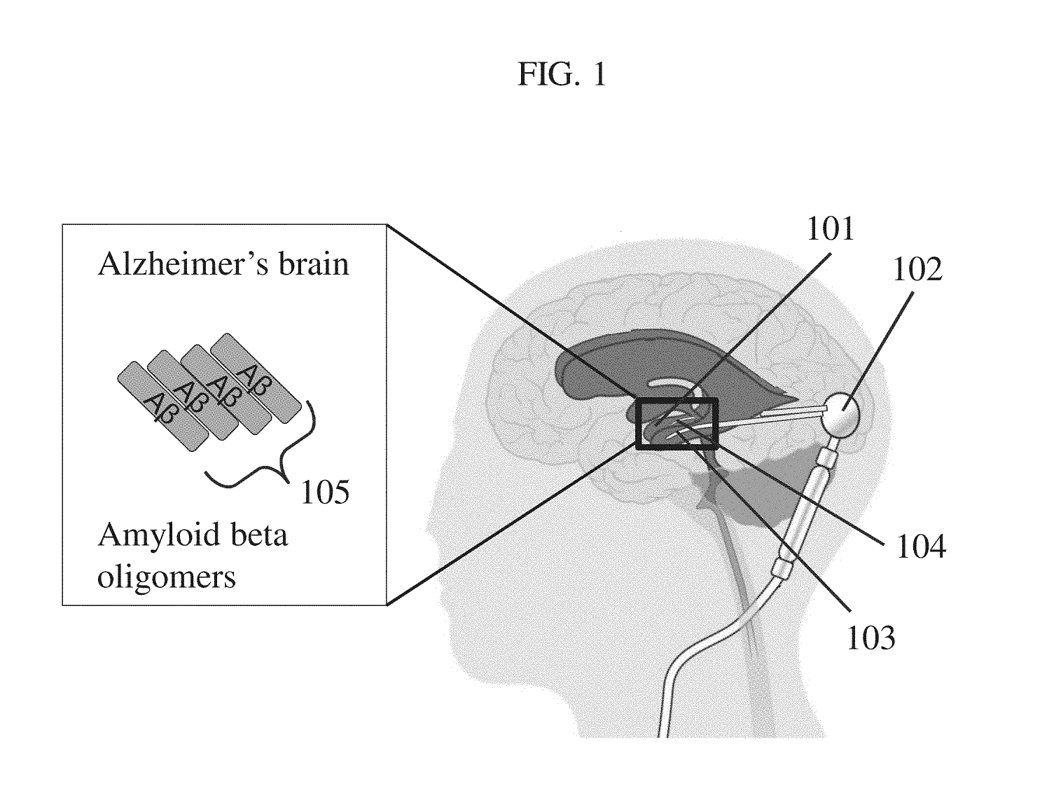

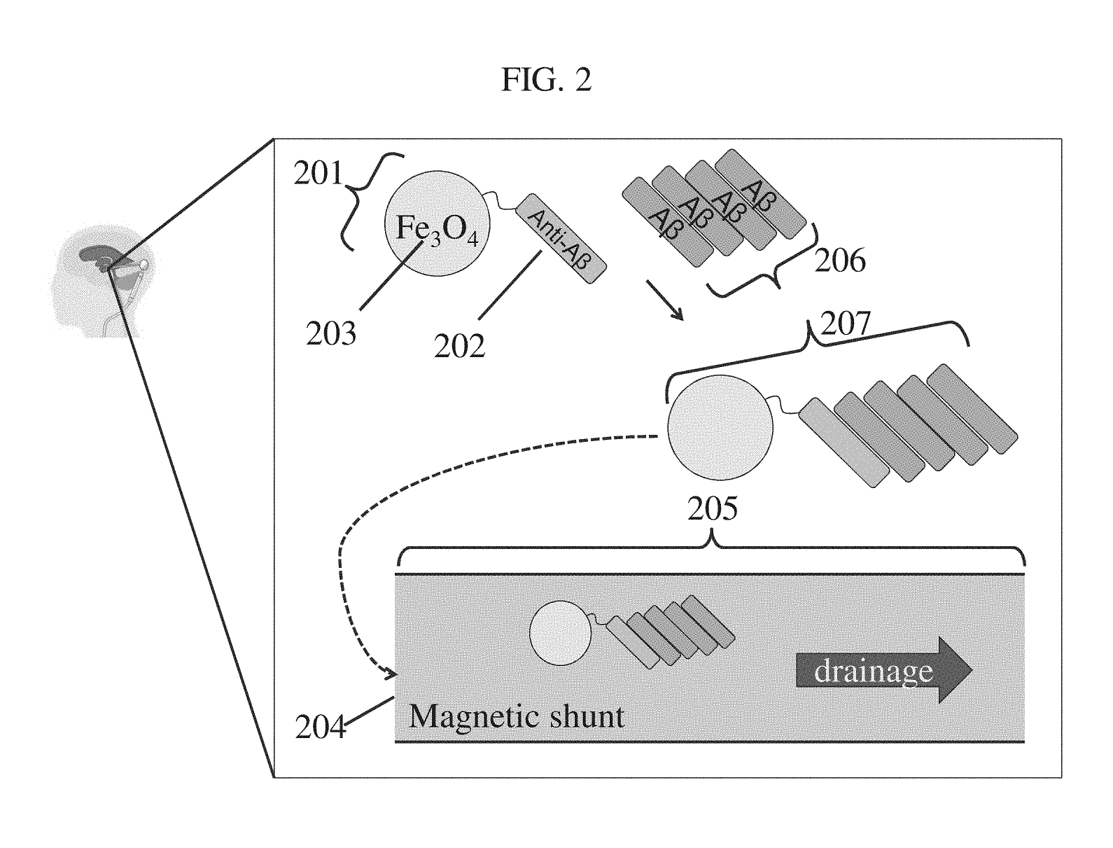

Disclosed herein are methods and compositions for removal of substances, such as amyloid beta oligomers. An example of method, compositions, and devices as disclosed herein to remove amyloid beta oligomers from brain tissue is depicted in FIG. 1 and FIG. 2. To target such substances, in some examples, the magnetic nanoparticles disclosed herein comprise a targeting agent that facilitates crossing the blood brain barrier.

### Magnetic Nanoparticle

### Composition

Disclosed herein are nanoparticles comprising a portion configured to couple the nanoparticle to a device. Such nanoparticles can comprise magnetic nanoparticles. The magnetic nanoparticles can comprise a magnetic element. Some suitable magnetic elements include, but are not limited to, ferrous, ferric oxide, ferromagnetic iron oxide, maghemite, gamma-Fe2O3, magnetite, Fe3O4, magnetite, lodestone, iron, nickel, cobalt, gadolinium, dysprosium, and aluminum. The magnetic element can be a permanent magnet or an element that has been transiently or permanently magnetized. Magnetic nanoparticles as disclosed herein can, in some examples, comprise superparamagnetic iron oxide nanoparticles, also known as SPIONS.

A magnetic nanoparticle may be a metal or metal oxide particle comprised of gold, silver, copper, iron, palladium, platinum, or a combination thereof. The metal nanoparticles may be comprised of a single composition, or may include a core composition and a coating composition. Metal nanoparticles that are spherical in shape and comprise a single composition may be referred to as a metal nanosphere. Nanoparticles having a metal coating on a semiconductor, dielectric, or metallic core may be referred to as core-shell nanoparticles. In one embodiment, when the metal nanoparticles are core-shell nanoparticles, the core may be composed of a semiconductor, metal, metal oxide or dielectric material. For example, the semiconductor material that provides the core composition may be silicon (Si) or silica (SiO₂). The coating composition may be composed of a metal, such as gold. Nanoparticles having a hollow interior are referred to as nanoshells. In some examples, the nanoparticle can comprise a magnetic core coated with a non-magnetic shell for use in attaching targeting agents.

In some examples, the magnetic nanoparticle comprises a precursor for α-Fe₂O₃, γ-Fe₂O₃ or related nanoalloy oxides with Fe after oxidization or for bcc-Fe or alloys-based Fe nanocomponents after reducing. The precursor based on iron oxide can be extended to other iron oxide based nanomaterials, including, but not limited to, MFe₂O₄, RFeO₃, and MRFeOₓ (M=Ba, Bi, Co, Cr, Cu, Fe, Mg, Mn, Ni, Ti, Y, Zn) (R=rare earth metal elements) nanomaterials, and iron oxide coated various nanomaterials. In some embodiments, nanomaterials are FeO₂ nanoparticles.

Non-limiting examples of the shape of a nanoparticle include substantially spherical, platelet, rod-shaped, needles, prisms, or any combination thereof In some cases, a nanoparticle has a dimension, such as a radius or longest axis, of 1000 nm or less. In some examples, the longest axis of a nanoparticle ranges from 1 nm to 5000 nm. In some cases, the longest axis of a nanoparticle is less than 1 nm, whereas in other examples, the longest axis is greater than 5000 nm. In some examples, the nanoparticle size in at least one dimension is within 0.1-1000 nm, and in some examples between 8 and 20 nm.

Magnetic nanoparticles used for biomedical applications can comprise a composite where particles of a magnetic component are coated with a non-magnetic coating, such as a polymeric shell. The overall size of the composite, referred to as the hydrodynamic diameter, can be different from the size of the core of magnetic crystals, which can be mainly responsible for the magnetism of the composite. Such a coating can be a polymeric shell consisting of a biocompatible polymer such as dextran, starch, polysaccharides, proteins, or polyethyleneglycols.

Disclosed herein are magnetic nanoparticles comprising a nanomaterial which includes a magnetic nanocomponent coated by a single or multiple layer(s) of non-toxic metal oxide(s), with or without inclusion of quantum dot materials; optionally comprising a bio-inert surface coating with or without addition of bioactive polymers or bio-molecules, depending on the different application purposes. In some examples, magnetic nanoparticles can be composed of aggregation or assembly at a coarser scale of smaller magnetic nanoparticles.

In some examples, magnetic nanoparticles are encapsulated inside liposomes, polymers, biopolymers or biomaterials. In some examples, they are encapsulated along with therapeutic or biochemical cargo. In some examples, the nanoparticles disclosed herein comprise a tagging element and/or targeting agent. Such elements or agents, as well as any other desired cargo or active agent, can be associated with the nanoparticle based on covalent interactions, non-covalent interactions, hydrophobic bonds, ionic interactions, or hydrogen bonds between the active agent or element and the outer surface of the nanoparticle or nanoparticle coating.

### Tagging Element

Disclosed herein are nanoparticles, such as magnetic nanoparticles, comprising a portion configured to bind a substance of interest. In some embodiments, the portion configured to bind a substance of interest comprises a tagging element. A tagging element facilitates binding of the nanoparticle to a target substance. A tagging element can comprise an antibody, polypeptide, ligand, polynucleic acid molecule, oligonucleotide, streptavidin, vitamin, biotin, adapter molecule, chemical, curcumin, or any combination thereof. Other suitable tagging elements can depend on the target substance and would be readily recognized by one of skill in the art. The tagging element can be selected based on the target substance. For example, the tagging element can comprise an antibody that specifically binds to a target substance. Additionally or alternatively, the tagging element can comprise a polypeptide sequence that specifically binds to a target substance. The tagging element can comprise an adaptor, such as streptavidin, that specifically binds a ligand-labeled target substance, such as a biotin labeled target substance. A target substance can be any substance disclosed herein. In some examples, the tagging element is an anti-amyloid beta antibody, anti-Tau antibody, amyloid beta peptide, or tau peptide.

The present disclosure provides tagging elements, such as antibodies or peptides. In some examples, the tagging element comprises a monoclonal antibody that specifically binds to an isolated polypeptide comprised of at least five amino acid residues of target of embodiments of the present invention, for example proteins or markers associated with Alzheimer's disease. These antibodies can find use in the diagnostic and therapeutic methods described herein.

An antibody for use in embodiments of the present invention may be any monoclonal or polyclonal antibody, as long as it can recognize the protein or substance of interest. Antibodies can be produced by using a protein of the present invention as the antigen according to a conventional antibody or antiserum preparation process. The present disclosure contemplates the use of both monoclonal and polyclonal antibodies. Selection and purification of monoclonal or polyclonal antibodies can be carried out according to any known method or its modification.

In some examples, known antibodies are used to target amyloid beta oligomers, such as NU-4.

Suitable antibodies can include monoclonal anti-amyloid beta antibodies such as those derived from clones 6E10, 4G8, 12F4, 11A5-B10, and 6F/3D.

Suitable antibodies can include polyclonal anti-amyloid beta antibodies such as AB2539.

Suitable antibodies can include anti-amyloid beta antibodies such as those derived from clone 20.1, NAB228, 6E10, M3.2, poly8029, poly18258, poly18268, 7-14-4, 11A50-B10, 12F4, 9C4/Amyloid, 1E11, 2H4, 4G8, 139-5, 1-11-3, 1G6, 29-6, 337.48, 5F3, B10AP, BA1-13, BA3-9, or other known clones.

Suitable antibodies can include anti-amyloid beta antibodies such as an anti-amyloid beta peptide 1-8 antibody derived from clone 1E11, 2H4. Such antibodies can be purified and can comprise a tag, such as biotin or HRP.

Suitable antibodies can include anti-amyloid beta antibodies such as an anti-amyloid beta peptide 1-16 antibody derived from clone 6E10, M3.2, or poly8029. Such antibodies can be purified and can comprise a tag, such as biotin or HRP.

Suitable antibodies can include anti-amyloid beta antibodies such as an anti-amyloid beta peptide 1-38 antibody derived from clone 7-14-4. Such antibodies can be purified and can comprise a tag, such as biotin or HRP.

Suitable antibodies can include anti-amyloid beta antibodies such as an anti-amyloid beta peptide 1-40 antibody derived from clone 11A50-B10. Such antibodies can be purified and can comprise a tag, such as biotin or HRP.

Suitable antibodies can include anti-amyloid beta antibodies such as an anti-amyloid beta peptide 1-42 antibody derived from clone 12F4. Such antibodies can be purified and can comprise a tag, such as biotin or HRP.

Suitable antibodies can include anti-amyloid beta antibodies such as an anti-amyloid beta peptide 1-43 antibody derived from clone 9C4/Amyloid. Such antibodies can be purified and can comprise a tag, such as biotin or HRP.

Suitable antibodies can include anti-amyloid beta antibodies such as an anti-amyloid beta peptide 17-24 antibody derived from clone 4G8. Such antibodies can be purified and can comprise a tag, such as biotin or HRP.

Suitable antibodies can include anti-amyloid beta antibodies such as an anti-amyloid beta peptide x-40 antibody derived from clone 139-5. Such antibodies can be purified and can comprise a tag, such as biotin or HRP.

Suitable antibodies can include anti-amyloid beta antibodies such as an anti-amyloid beta peptide x-42 antibody derived from clone 1-11-3. Such antibodies can be purified and can comprise a tag, such as biotin or HRP.

Suitable antibodies can include antibodies generated against amyloid beta precursor or a fragment thereof. In some examples, a suitable antibody can be generated against amyloid beta peptide comprising amino acids 1-42 (SEQ ID NO 1: DAEFRHDSGYEVHHQKLVFFAED VGSNKGAIIGLMVGGVVIA).

Suitable antibodies can include tagged anti-amyloid beta antibodies such as antibodies comprising a biotin, HRP, fluorophore, Alexa Fluor 488, GFP, or other reporter or signaling tag known in the art.

A tagging element can comprise an amyloid beta binding compound. An amyloid beta binding compound can comprise Thioflavin-S, Thioflavin-T, Congo Red, curcumin, curcumin derivatives, methoxy-O4, chrysamine G, chyrsamine G derivatives, X-34, BSB, clioquinol, Texas red, Texas red derivatives, or other suitable compounds known in the art.

A tagging element can comprise a plaque detecting compound. A plaque-detecting compound can comprise curcumin, curcumin derivatives, Pittsburg compound-B (PiB), Florbetapir (18F), AMYViD, florbetapir-fluorine-18, 18FAV-45, 18F-Fluorbetaben, thioflavin, thioflavin derivatives, Congo red, Congo red derivatives, DDNP (1, 1-dicyano-2-[6(dimethylamino)naphthalene-2-yl]propene, or other suitable compounds known in the art.

### Targeting Ligand

Disclosed herein are nanoparticles, such as magnetic nanoparticles comprising a portion configured to facilitate localization or translocation of the nanoparticle to an environment. In some embodiments, such a portion comprises a targeting ligand. A targeting ligand can facilitate targeting of the nanoparticle to the desired location. A targeting ligand can facilitate transport of the nanoparticle to the desired location. In some examples, a targeting ligand facilitates targeting or transport of a nanoparticle to a desired tissue type, cell type, or subcellular location. In some examples, the targeting ligand facilitates receptor mediate transport. Receptor mediated transport can, in some examples, transport the nanoparticle across the blood brain barrier. The targeting ligand can be transferrin or insulin. In some examples, the targeting ligand is capable of binding to a receptor, such as a transferrin receptor, insulin receptor, or a low density lipoprotein receptor. Suitable targeting ligands are also described in Lajoie and Shusta 2015, Annu. Rev. Pharmacol. Toxicol. 2015, 55:613-31.

In some cases, a targeting ligand comprises an anti-transferrin receptor antibody that binds a transferrin receptor. In some examples, anti-transferrin receptor antibody is used as a targeting ligand for slow release of a nanoparticle across the blood brain barrier.

In some cases, a targeting ligand comprises a transferrin receptor antibody that binds a transferrin receptor. In some examples, anti-transferrin receptor antibody is used as a targeting ligand for slow release of a nanoparticle across the blood brain barrier.

In some cases, a targeting ligand comprises a transferrin receptor binding peptide. For example, a peptide comprising HAIYPRH (SEQ ID NO: 2), CHAIYPRH (SEQ ID NO: 3), THRPPMWSPVWP (SEQ ID NO: 4, transferrin receptor targeting peptide), or a T7 tagged peptide. In some examples, transferrin receptor binding peptides are useful for fast release of a nanoparticle across the blood brain barrier.

In some cases, a targeting ligand comprises transferrin, which competes with endogenous transferrin for binding to a transferrin receptor.

In some cases, a targeting ligand comprises ApoE, COG133, COG112, or other apolipoprotein mimetic peptide. Apolipoprotein mimetic peptides, such as ApoE, COB133, and COG112, can bind a LDL receptor and can be used for translocation across the blood brain barrier. An exemplary COG133 peptide comprises the amino acid sequence LRVRLASHLRKLRKRLL (SEQ ID NO: 5).

In some cases, a targeting ligand comprises a peptide such as Angiopep-2, for example, a peptide comprising the amino acid sequence TFFYGGSRGKRNNFKTEEY (SEQ ID NO: 6). An Angiopep-2 can bind LRP of the LDL receptor family to facilitate translocation across the blood brain barrier. In some examples, Angiopep-2 is used for fast release and high transcytosis rates. In some cases, translocation via Angiopep-2 and an LDL receptor mediated pathway is faster than translocation via a transferrin receptor mediated pathway.

In some cases, a targeting ligand comprises a leptin, anti-leptin or an anti-ObR polyclonal or monoclonal antibody. Leptin or an anti-ObR antibody can bind to ObR to facilitate translocation across the blood brain barrier. In some examples, translocation is achieved via micropinocytosis or transcytosis.

In some cases, a targeting ligand comprises Diphteriatoxin, such as the nontoxic CRM197. Diphtheriatoxin can bind to the membrane-bound Heparin binding-EGF precursor (DT receptor) exposed on the endothelia, neurons, glia cells, or other NCS cells. Translocation across the blood brain barrier mediated by such a ligand and receptor can be facilitated via transvascular and brain cell-specific receptor uptake after binding to HB-EGF precursor. Translocation by this method can be specific to cells comprising a membrane-bound HB-EGF precursor.

In some cases, a targeting ligand comprises opioid peptides, such as Enkephalins. In some examples, such opioid peptides are glycosylated. Non-limiting examples of opioid peptides include G7-peptide, and an MMP-2200 mimetic with Tyr replaced by Phe. Opioid peptides such as G7-peptide can bind to encephalin receptors and facilitate translocation across the blood brain barrier via micropinocytosis or via an encephalin transporter.

In some cases, a targeting ligand comprises a rabies virus RVG peptide. A RVG peptide can bind to an N-acetylcholine receptor and can facilitate translocation of the blood brain barrier via receptor mediate transcytosis. In some examples, the RVG peptide is expressed in exosomes to avoid IL-6 production. In some examples, RVG-9R peptide delivers nucleic acid cargo, such as siRNA, to neuronal cell.

In some cases, a targeting ligand comprises a tetanus toxin, such as G23 peptide. Such Tetanus toxin peptides can bind a ganglioside and can facilitate translocations across the blood brain barrier via transcytosis. In some examples, tetanus toxin peptides facilitate translocation of polymerosomes. In some examples, translocation by tetanus toxin peptides leads to neuron specific accumulation of the associated cargo.

In some cases, a targeting ligand comprises a exocyclic peptide, such as MiniAp-4 or other mellitin derived peptide. Such exocyclic peptides can be protease stable.

In some cases, targeting ligands facilitate targeting and/or transport of a nanoparticle to a tissue or cell type of interest. Tissues or cell types of interests include, but are not limited to, brain, cerebrospinal fluid, temporal horn of the brain, other specific brain lobes or locations, kidney, lungs, liver, blood, skin, and other organ or tissue types within a body and any specific cell types comprised therein.

In some cases, targeting ligands facilitate targeting or transport of a nanoparticle to a subcellular domain of interest. Subcellular domains of interest include, but are not limited to, cytoplasm, periplasm, nucleus, mitochondria, and cellular membrane.

### Heat Generation

Disclosed herein are nanoparticles, such as magnetic nanoparticles, configured to generate heat in response to a magnetic stimuli. Examples of materials that may be activated to generate heat using a magnetic stimuli, such as an alternating electromagnetic field, include magnetite nanoparticles (Fe₃O₄). Other examples of materials that generate heat when subjected to an electromagnetic field include composite particles of cobalt (Co), lanthium (La), strontium (Sr) and manganese (Mn). In some embodiments, these materials have superparamagnetic properties, e.g., when the individual particle size is less than 15 nm and composed of a single magnetic domain. Superparamagnetism is a form of magnetism that appears in nanoparticles having a single magnetic domain, in which the magnetism can randomly change direction under the influence of an alternating magnetic field. Nanoparticles having a composition that is activated by magnetic stimuli can be inductively heated by a magnetic field generated by an alternating current. Heating can be attributed to friction of the particle rotating in the magnetic field or to Neel relaxation where energy applied to the particle, by the alternating magnetic field, allows the magnetic moment in the particle to overcome the energy barrier. This energy is then dissipated as heat when the particle moment relaxes to its equilibrium orientation. It is noted that larger particles of multiple superparamagnetic particles can also be synthesized.

In some examples, in response to a stimuli of a magnetic field, the temperature of the nanoparticles increases or decreases by +/−1000° C. from an ambient temperature that ranges from about 20° C. to about 40° C. In some examples, in response to a stimuli of a magnetic field, the temperature of the nanoparticles increases or decreases by +/−100° C. from an ambient temperature that ranges from about 35° C. to about 40° C.

In some embodiments, when exposed to alternate magnetic fields, the nanoparticles absorb radio frequency and generate heat that is sufficient to degrade and/or destroy a bound substance and/or kill a bound cell. For example, when the nanoparticles are selectively bound to an amyloid beta fibril, they can selectively degrade the fibril.

### Imaging

Disclosed herein are nanoparticles, such as magnetic nanoparticles, comprising a tagging element by which the magnetic nanoparticle targets or tags a substance of interest. These nanoparticles are magnetically active and hence can be detected by conventional MRI. The agents can serve as contrast agents and selective targeting agents.

In some examples, magnetic nanoparticles disclosed herein comprise a contrast agent for imaging, such as by X-Ray, computer tomography (CT) imaging, or MRI imaging. In some examples, nanoparticles comprise tagging elements, such as nucleic acids, PNAs, peptides, proteins, and/or antibodies, which target the nanoparticles to a region or substance of interest, such as amyloid beta fibrils. Nanoparticles disclosed herein can be used in drug screening applications or research, such as imaging in animal models, structural studies, DNA-protein binding interactions, or protein capture. Magnetic nanoparticles as disclosed herein can be used in in vivo or in vitro imaging methods, for example, fluorescence microscopy, MRI imaging, and/or AFM.

Further provided are diagnostic assays for Alzheimer's disease. In some examples, assays utilize magnetic nanoparticles as disclosed herein comprising a tagging element, such as an anti-amyloid beta antibody. Utilizing the tagging elements, such as antibody functionality, facilitates delivery of contrast agents to a site of interest, such as an amyloid beta oligomer bound neurite. Thus, embodiments of the present disclosure provide possible early detection of Alzheimer disease using a clinical imaging system such as MRI.

### Removal Device

Disclosed herein are magnetic field generating devices, which include removal devices. In some examples, the magnetic field generating devices are configured to accumulate magnetic nanoparticles such as those disclosed herein. In some examples, the magnetic field generating devices are configured to remove magnetic nanoparticles such as those disclosed herein, from an environment. For example, the removal device can remove said magnetic nanoparticles from a tissue or fluid, such as brain, cerebrospinal fluid, lung, kidney, liver, skin, or blood.

### Conduit

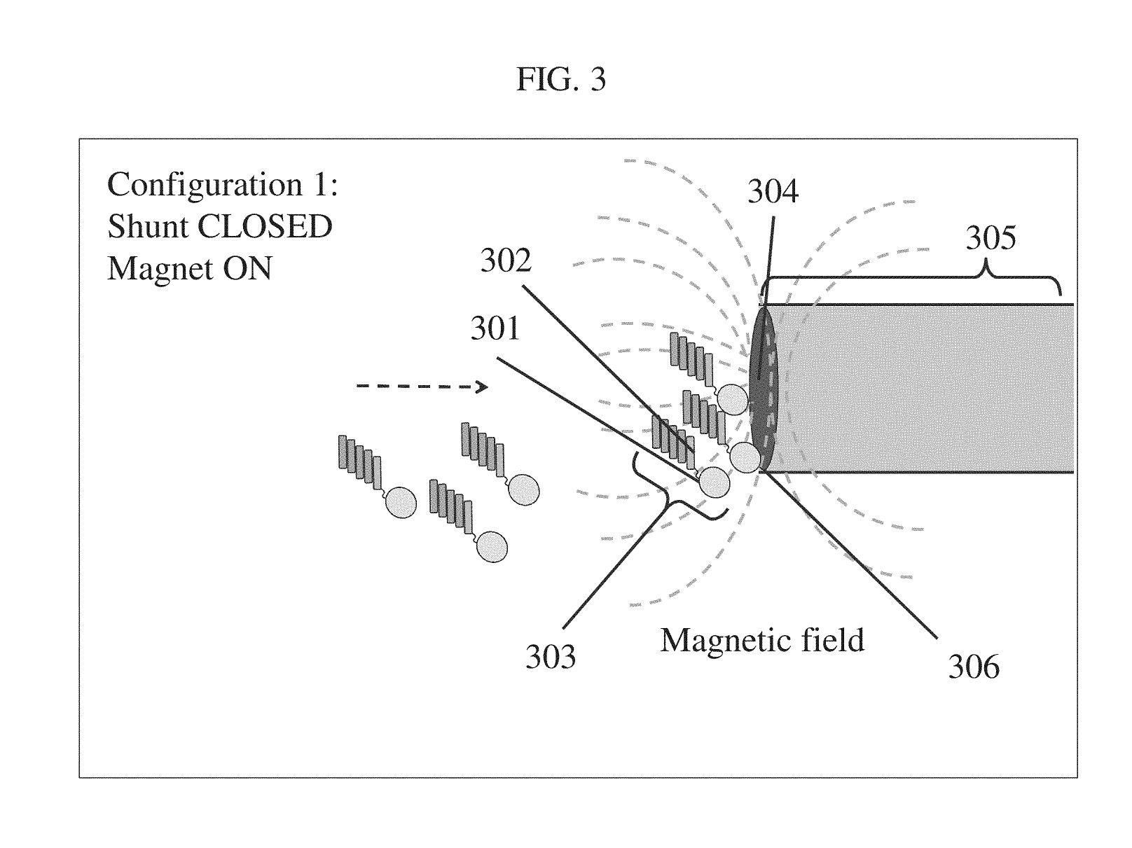

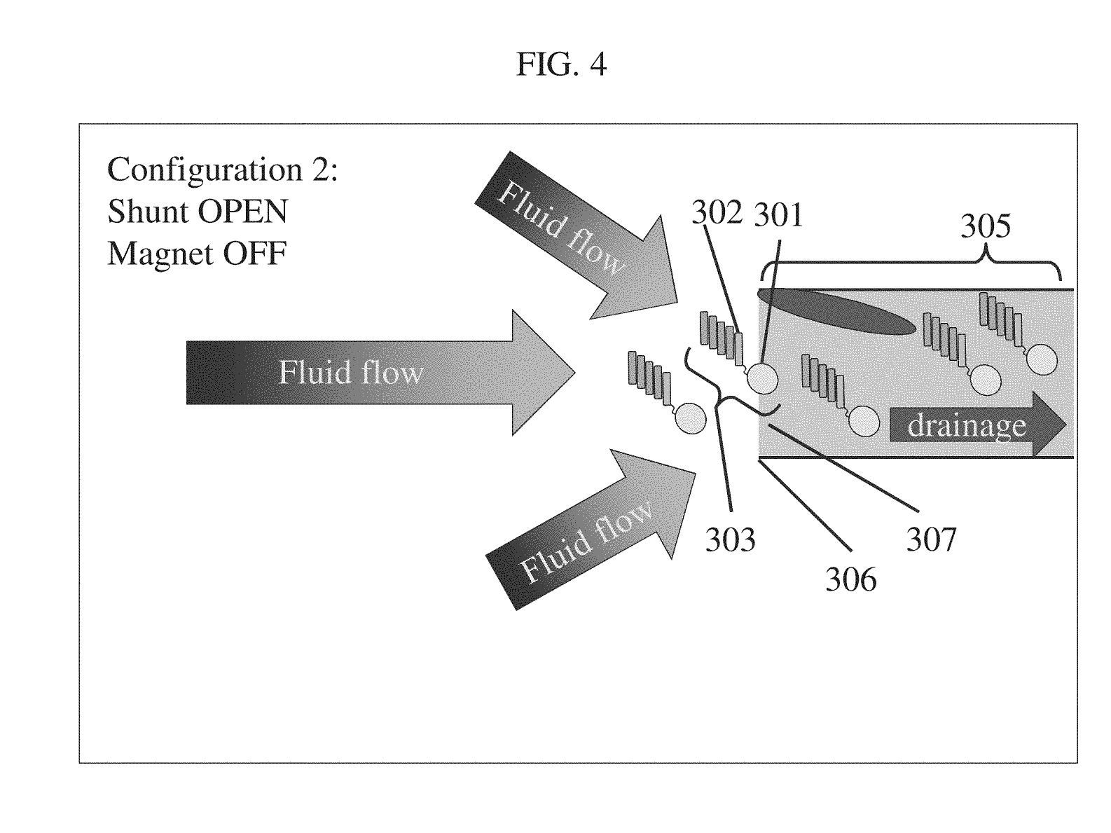

Disclosed herein are magnetic field generating devices comprising a conduit. In some examples, removal of magnetic nanoparticles occurs through the conduit. In some examples, the conduit comprises a distal end which comprises an opening. In some such examples, the conduit is configured to have a first configuration and a second configuration. For example, in a first configuration, the distal end is closed such that no or limited fluid and/or particles can enter or be received into the conduit; while in a second configuration, the distal end is open such that fluid and/or particles can enter or be received into the conduit. An example of such a first configuration is depicted in FIG. 3, and an example of such a second configuration is depicted in FIG. 4.

### Magnetic Field

Disclosed herein are magnetic field generating devices configured to emit a magnetic field. In some cases, the magnetic field can be turned on or off. In some cases, the magnetic field is adjustable to increase or decrease the strength of the magnetic field. In some examples, the magnetic field comprises a gradient of magnetic field strengths.

In some examples, a magnetic field is generated by one or more magnets. A magnetic field can effectively be turned on by orienting one or more magnets to point in the same direction. A magnetic field can be effectively turned off by orienting two or more magnets to point in opposite directions, thereby cancelling out the magnetic field generated by each magnet. The orientation of one or more magnets can be controlled manually or automatically using a device such as a timer mechanism.

In some examples, magnetic particles accumulate or collect at a site of magnetic field emission. In some examples, the magnetic field is emitted from a distal end of a conduit, such that magnetic particles magnetically couple with the distal end.

### Valve

Disclosed herein are magnetic field generating devices comprising a valve. In some examples a valve can be open, closed, or in a range of semi-open positions. The valve can control pressure within a section of the device, such as a conduit. In some examples, flow of fluid or particles into a conduit is controlled by adjusting the pressure within the conduit compared to the surrounding environment. For example, decreasing the pressure within the conduit will generate a pressure gradient that favors flow into the conduit. Alternatively, matching the pressure of the conduit to the surrounding environment removes a pressure gradient and thereby decrease flow into the conduit. Increasing the pressure within the conduit will generate a pressure gradient that favors flow from the conduit into the surrounding environment. Therefore, adjusting the pressure level within the conduit can control the flow rate into the conduit by controlling the presence and level of a pressure gradient between the conduit and the surrounding environment.

### Medical Device

Disclosed herein are magnetic field generating devices that comprise a medical device. As non-limiting examples, the magnetic field generating device comprises a shunt, catheter, implant, intermittent device, or a device comprising electrode stimulators. In some examples, the magnetic field generating device comprises two or more shunts or catheters. In some examples, the magnetic field generating device comprises two or more conduits with distal ends as disclosed herein.

Exemplary methods for preparing devices include plastics extrusion, casting, and molding. Plastics extrusion is a process in which raw plastic material is melted and formed into a continuous profile through a two-dimensional die. Casting is a manufacturing process by which a liquid material is usually poured into a mold, which contains a hollow cavity of the desired shape, and then allowed to solidify. Injection molding is a process for producing parts from both thermoplastic and thermosetting plastic materials. Material is fed into a heated barrel, mixed, and forced into a three dimensional mold cavity where it cools and hardens to the configuration of the mold cavity.

The mold, casting or extrusion die of the above described methods may have a geometry that provides at least one component of any magnetic field generated device disclosed herein. In some examples, the mold, casting or extrusion die is selected to form at least one component of a shunt, catheter, artificial joint, dental implant, cosmetic implant or other medical implant or intermittent device.

### Target Substances

Disclosed herein are methods and compositions for the removal of a target substance. A target substance can be any entity for which removal is desired. In some cases, a target substance comprises, as non-limiting examples, biological material, such as polynucleic acids, nucleic acid molecules, polypeptides, proteins, misfolded proteins, prions, lipids, fatty acids, cholesterol, and bile components; elements such as calcium; vesicles; lipid bilayer; cells (e.g., cancer cells, inflammatory cells); and/or any molecule generated, metabolized, or secreted by a cell. In some cases, a target substance comprises, as non-limiting examples, a toxic, pathogenic, or infectious entity, such as a virus, viral particle, parasite, archaea, bacterium, microorganism, toxic compound, chemical, toxic element, lead, cancer cell, and/or prion. In some cases, a target substance comprises, as non-limiting examples, an accumulated plurality of any substance or substance disclosed herein. For example, a target substance comprises Tau protein oligomers, amyloid beta oligomers/fibrils, bacterial biofilm, calcium deposit, cholesterol plaque, bile stone, and/or kidney stone.

### Magnetic-based Removal of Substances

Disclosed herein are methods and compositions for the removal of a target substance. A target substance can be tagged by a magnetic nanoparticle as disclosed herein. In some examples a magnetic particle comprises a tagging element that is capable of targeting the magnetic nanoparticle to the substance. A tagging element can comprise any tagging element disclosed herein. For example, a tagging element comprises an anti-amyloid beta antibody.

In some examples, a magnetic nanoparticle comprises a targeting ligand that facilitates targeting or transport of the nanoparticle to a desired location. For example, a targeting ligand comprises transferrin, which facilitates translocation across the blood brain barrier by coupling with a transferrin receptor. A targeting ligand can comprise any targeting ligand that facilitated receptor-mediate transport across the blood brain barrier. Additionally or alternatively, a targeting ligand can comprise any targeting ligand disclosed herein.

In some examples, a magnetic nanoparticle comprising a tagging element binds to a target substance, thereby generating a nanoparticle-substance complex. In some examples, the tagging element is an anti-amyloid beta antibody and the substance is amyloid beta protein or fibril.

In some examples, a magnetic field generating device is positioned in proximity to a substance for which removal is desired. A magnetic field generating device includes any magnetic field generating device disclosed herein. For example, a magnetic field generating device comprises a shunt. Such a shunt can comprise a conduit with a distal end capable of being open, closed, or a range of open positions. In some examples, the device comprises two shunts capable each comprising a conduit. In such examples, each conduit can be independently opened and closed, or both conduits can be opened and closed together, either concurrently or sequentially. In some examples, the device comprises two shunts, and each one is positioned into a temporal horn of the brain. An example of such a dual shunt device is depicted in FIG. 1.

In some examples, the distal end of a conduit emits a magnetic field such that it is able to magnetically couple with a magnetic nanoparticle as disclosed herein. In many examples, the emitted magnetic field is able to be turned on, turned off, increased, and/or decreased. In some examples, the magnetic field is turned on when the distal end of the conduit is closed, such that magnetic particles are collected or accumulated at the distal end, though they may not be able to enter or be received into the conduit. In some examples, the magnetic field is turned off when the distal end of the conduit is opened, such that magnetic particles are released from the distal end, and therefore able to enter or be received into the conduit.

In some examples, the flow rate of fluid or particles into the conduit of a device is controlled by a valve as disclosed herein. Such a valve can control the pressure within a conduit relative to the surrounding environment and therefor control the rate and direction of flow.

### Kits and Systems

In some embodiments, the present disclosure provides kits for using in research, diagnostic and therapeutic applications. In some embodiments, kits include components necessary, sufficient and/or useful in performing the methods of embodiments of the present invention.

In some embodiments, kits include substance-targeting magnetic nanoparticles and magnetic field generating device, along with any controls, buffers, reagents, administration tools, etc.

Kits may further comprise appropriate controls and/or detection reagents. Any one or more reagents that find use in any of the methods described herein may be provided in the kit.

In some embodiments, the present invention provides systems for use in targeting and treating Alzheimer's disease. In some embodiments, systems comprise the above described components and a device for generating a magnetic field for use in therapy or removal of Alzheimer's plaques, fibrils, or misfolded protein oligomers

Referring to the figures, FIG. 1 depicts an embodiment of a magnetic field generating device as described herein. In this example, a dual-shunt 102 comprising a first distal end 103 and second distal end 104 is positioned near tissue 101 in the temporal horns of a human brain, which comprises a substance of interest 105, in this case amyloid beta oligomers. In the depicted embodiment, the magnetic field generating device is a dual-shunt 102. In alternate embodiments, the magnetic field generating device comprises a single shunt, a catheter, a dual-catheter, an implant, an intermittent device, artificial joint, dental implant, cosmetic implant, or other medical device. In some examples the device comprises one conduit. In some examples, the device comprises two or more conduits. In some examples, the conduit is configured to comprise an open and a closed position, and in some of these cases, switching between the open and closed configurations can be done manually or automatically. In some examples, the device is configured to generate a magnetic field. In some examples the magnetic field can be turned on and turned off In some examples, the magnetic field can be adjusted so as to increase or decrease the magnetic field strength. In some examples, adjusting or turning the magnetic field on or turning the magnetic field off can be done manually or automatically. In some examples the device further comprises a valve. In some examples, the valve controls pressure within a conduit such as to control flow of fluid and/or particles into or out of the conduit. In the depicted embodiment, the magnetic field generating device is positioned within tissue 101 within the temporal horns of the brain. In alternate embodiments, the device can be positioned within other sections of the brain, lungs, kidney, bladder, blood, skin, other and other bodily organ or tissue within a mammal. In some examples, the device is positioned within tissue or fluid surrounded by tissue or cell membrane. In the depicted example, the substance of interest is an amyloid beta oligomer 105. In alternate examples, the substance of interest can be as non-limiting examples, biological material, such as polynucleic acids, nucleic acid molecules, polypeptides, proteins, misfolded proteins, prions, lipids, fatty acids, cholesterol, bile components, elements such as calcium, vesicles, lipid bilayer, cell, cancer cell, inflammatory cell, or any molecule generated, metabolized, or secreted by a cell. A substance of interest can comprise, as non-limiting examples, a toxic, pathogenic, or infectious entity, such as a virus, viral particle, parasite, archaea, bacterium, microorganism, toxic compound, chemical, toxic element, lead, cancer cell, or prion. A substance of interest can comprise, as non-limiting examples, an accumulated plurality of any substance or substance disclosed herein. For example, a substance of interest can comprise as non-limiting examples, Tau protein oligomers, amyloid beta oligomers or fibrils, bacterial biofilm, calcium deposit, cholesterol plaque, bile stone, or kidney stone.

FIG. 2 depicts an embodiment of a nanoparticle and a device as disclosed herein, said nanoparticle 201 comprising a) anti-amyloid beta antibody 202 configured to bind to an amyloid beta oligomer 206, thereby generating a nanoparticle-amyloid beta complex 207, and b) an Fe₃O₄ core 203 configured to couple the nanoparticle to magnetic field generating shunt comprising a conduit 205 with a distal end 204 configured to emit the magnetic field. In the depicted example, the magnetic nanoparticle has a Fe₃O₄ core 203. In other embodiments, the magnetic nanoparticle comprises ferrous, ferric oxide, ferromagnetic iron oxide, maghemite, gamma-Fe2O3, magnetite, Fe3O4, magnetite, lodestone, iron, nickel, cobalt, gadolinium, dysprosium, aluminum, or a combination thereof. The magnetic nanoparticle can comprise a magnetic element. The magnetic element can be a permanent element or a transiently magnetized element. The nanoparticle can further comprise a coating to which additional agent or elements are attached. An example of such an element is the anti-amyloid beta antibody 202 depicted in FIG. 2. In other embodiments, the nanoparticle can comprise a portion configured to couple the nanoparticle to a substance of interest. Such a portion can comprise a tagging element. A tagging element can be an antibody, peptide, ligand, polynucleic acid molecule, oligonucleotide, streptavidin, vitamin, biotin, adapter molecule, chemical, curcumin, or any combination thereof. An antibody can be a monoclonal or polyclonal antibody. In some embodiments, the tagging element can comprise any tagging element disclosed herein. In the depicted example, the substance of interest is an amyloid beta oligomer 206. In alternate examples, the substance of interest can be as non-limiting examples, biological material, such as polynucleic acids, nucleic acid molecules, polypeptides, proteins, misfolded proteins, prions, lipids, fatty acids, cholesterol, bile components, elements such as calcium, vesicles, lipid bilayer, cell, cancer cell, inflammatory cell, or any molecule generated, metabolized, or secreted by a cell. A substance of interest can comprise, as non-limiting examples, a toxic, pathogenic, or infectious entity, such as a virus, viral particle, parasite, archaea, bacterium, microorganism, toxic compound, chemical, toxic element, lead, cancer cell, or prion. A substance of interest can comprise, as non-limiting examples, an accumulated plurality of any substance or substance disclosed herein. For example, a substance of interest can comprise as non-limiting examples, Tau protein oligomers, amyloid beta oligomers or fibrils, bacterial biofilm, calcium deposit, cholesterol plaque, bile stone, or kidney stone.

FIG. 3 depicts an embodiment of a method for removing a substance using a nanoparticle and a device as disclosed herein, said device being depicted in a first configuration wherein said device comprises a conduit 305 comprising a closed 304 distal end 306 emitting a magnetic field. Further depicted are nanoparticles comprising a) a first portion 302 comprising a tagging element configured to bind to the substance, thereby generating a nanoparticle-substance complex 303, and b) a second portion 301 capable of magnetically coupling with the magnetic field generating device, such that the nanoparticle-substance complexes 303 accumulate at the distal end 306 of the device. In the depicted embodiment, the device is a shunt comprising a conduit 305 comprising a closed 304 distal end. In some examples, closing of the distal end of the shunt can be manual or automatic. In the depicted example, the nanoparticle-substance complex 303 comprises a magnetic nanoparticle comprising an Fe₃O₄ core and an anti-amyloid beta antibody bound to an amyloid beta oligomer. In other embodiments, the magnetic nanoparticle can comprise any magnetic element disclosed herein. In other embodiments, the magnetic nanoparticle can comprise any tagging element disclosed herein configured to bind to any substance of interest or target substance and disclosed herein. In some examples, the nanoparticle further comprises a targeting ligand as disclosed herein. For example, the nanoparticle can comprise transferrin capable of binding to a transferrin receptor, thereby facilitating translocation across the blood brain barrier. Translocation of the blood brain barrier can be achieved in other embodiments using any other targeting ligand or method described herein. In the depicted configuration, the magnetic field is on and the shunt distal end is closed 304. In some embodiments, the magnetic field is turned on and the shunt distal end is closed simultaneously. In other embodiments, the magnetic field is turned on and the shunt distal end is closed sequentially. In some examples, turning on the magnetic field and/or closing the distal end of the conduit or shunt is done manually or automatically. In the depicted example, the nanoparticle-substance complexes accumulate at the distal end of the conduit of the shunt 306. In alternate embodiments, the nanoparticle-substance complexes can collect in a collection area. Additionally or alternatively, the nanoparticle-substance complexes can accumulate along any region of the device configured to emit a magnetic field capable of magnetically coupling with the nanoparticle and/or configured to accumulate or collect the nanoparticle. In the depicted example, the distal end of the conduit is closed 304. In some examples, no fluid and/or particles can enter the conduit when the distal end is closed. In some examples, limited or controlled levels of fluid and/or particles can enter the conduit when the distal end is closed. In the depicted example, a magnetic field is emitted from the distal end of the conduit 306. In some examples, the strength of the magnetic field can be adjusted so as to control the range or area within which the device is able to magnetically couple with magnetic nanoparticles. For example, the magnetic field strength can be dampened in order to collect or magnetically couple nanoparticles from a smaller area, or the magnetic field strength can be increased in order to collect or magnetically couple with magnetic nanoparticles over a wider area. Increase or decrease of the magnetic field strength can be done manually or automatically.

FIG. 4 depicts an embodiment of a method for removing a substance using a nanoparticle and a device as disclosed herein, said device being depicted in a second configuration wherein said device comprises a conduit 305 comprising an open 307 distal end 306, wherein the magnetic field has been turned off. Further depicted are nanoparticles comprising a) a first portion 302 comprising a tagging element configured to bind to the substance, thereby generating a nanoparticle-substance complex 303, and b) a second portion 301 capable of releasing from the magnetic field generating device when the magnetic field is off, such that the nanoparticle-substance complexes 303 are free to enter the 306 of the conduit 305 of the device, thereby being removed. In the depicted embodiment, the device is a shunt comprising a conduit 305 comprising an open 307 distal end. In some examples, opening of the distal end of the shunt can be manual or automatic. In the depicted example, the nanoparticle-substance complex 303 comprises a magnetic nanoparticle comprising an Fe₃O₄ core and an anti-amyloid beta antibody bound to an amyloid beta oligomer. In other embodiments, the magnetic nanoparticle can comprise any magnetic element disclosed herein. In other embodiments, the magnetic nanoparticle can comprise any tagging element disclosed herein configured to bind to any substance of interest or target substance and disclosed herein. In some examples, the nanoparticle further comprises a targeting ligand as disclosed herein. For example, the nanoparticle can comprise transferrin capable of binding to a transferrin receptor, thereby facilitating translocation across the blood brain barrier. Translocation of the blood brain barrier can be achieved in other embodiments using any other targeting ligand or method described herein. In the depicted configuration, the magnetic field is off and the shunt distal end is open 307. In some embodiments, the magnetic field is turned off and the shunt distal end is opened simultaneously. In other embodiments, the magnetic field is turned off and the shunt distal end is opened sequentially. In some examples, turning off the magnetic field and/or opening the distal end of the conduit or shunt is done manually or automatically. In the depicted example, the nanoparticle-substance complexes accumulated at the distal end of the conduit of the shunt 306 are released when the magnetic field is off In alternate embodiments, the nanoparticle-substance complexes can be released from a collection area. Additionally or alternatively, the nanoparticle-substance complexes can be released from an area along any region of the device configured to emit a magnetic field capable of magnetically coupling with the nanoparticle and/or configured to accumulate or collect the nanoparticle when the magnetic field is on. In the depicted example, the distal end of the conduit is open 307. In some examples, fluid and/or particles can enter the conduit when the distal end is open. In some examples, limited or controlled levels of fluid and/or particles can enter the conduit when the distal end is closed. In some embodiments, the device further comprises a valve configured to control the pressure within the conduit, thereby controlling, either manually or automatically, the flow rate of fluid and/or particles into the conduit. In the depicted example, the nanoparticle-substance complex 303 is removed from the environment or tissue when the device is in the depicted configuration. In some examples, the nanoparticle-substance complex is removed from a tissue or body through the conduit 305. In some examples, the nanoparticle-substance complex are collected within a region of the conduit or device configured to receive and collect the nanoparticle-substance complexes.

### EXAMPLES

### Example 1

### Magnetic Nanoparticle with Amyloid Beta Antibody

A magnetic nanoparticle is coated with anti-amyloid beta antibodies and transferrin. The anti-amyloid beta antibodies allow the nanoparticle to bind to amyloid beta. The transferrin allows the nanoparticle to couple with a transferrin receptor. The magnetic property of the nanoparticle is facilitated by a core of iron (III) oxide (gamma-Fe2O3) and allows the nanoparticle to bind to a device emitting a magnetic field.

### Example 2

### Magnetic Removal Device Comprising a Shunt

A shunt configured to be placed in a temporal horn of the brain has a distal end that is capable of emitting a magnetic field. The distal end can be opened or closed. When the distal end is open, the conduit of the shunt can be accessed and the shunt is able to receive fluids or particles into the conduit section. When the distal end is closed, fluids or particles cannot enter the conduit section of the shunt.

The generated magnetic field that is emitted by the distal end is able to be turned on, turned off, as well as adjusted to increase or decrease the magnetic field strength. When the magnetic field is on, the distal end of the conduit is closed. When the magnetic field is off, the distal end of the conduit is open.

The shunt also has a valve that is able to control flow rate into the conduit section of the shunt by increasing or decreasing the pressure gradient between the conduit and the surrounding environment.

### Example 3

### Magnetic Removal Device Comprising a Dual-shunt

A medical device as described in Example 2 is configured to have two shunts. The dual-shunt is configured such that a shunt can be placed in each temporal horn in a brain. The open/closed state and the flow rate of each shunt are controlled individually between the two shunts.

### Example 4

### Removal of Amyloid Beta Fibrils from Brain Tissue

Magnetic nanoparticles as described in Example 1 are administered to an Alzheimer's patient. The transferrin allows coupling of the nanoparticle with transferrin receptors in the brain, thereby allowing the nanoparticle to cross the blood brain barrier through receptor-mediated transfer. Once in the temporal horn of the brain, the anti-amyloid beta antibodies facilitate binding of the nanoparticle to amyloid beta fibrils. Binding of the nanoparticle decreases the growth of the fibrils by blocking further amyloid beta accumulation.

A magnetic field generating dual-shunt as described in Example 3 is inserted into the brain of the Alzheimer's patient. One shunt is placed into each temporal horn. The magnetic field is turned on and the nanoparticles bound to the amyloid beta fibrils magnetically couple with the distal end of the shunt. The nanoparticles accumulate at the distal end of the shunt but do not enter the conduit of the shunt since the shunt is closed.

After accumulation of the nanoparticle-amyloid beta complexes, the magnetic field is turned off while the distal end of the conduit is opened. The valve of each shunt is adjusted to decrease the pressure within the shunt, thereby generating a pressure gradient that favors the flow of cerebrospinal fluid into the shunt. The flow rate is controlled by the valve.

While the magnetic field is off and the conduit is opened, the accumulated nanoparticle-amyloid beta complexes are released from the distal end and are drawn into the conduit by the flow of cerebrospinal fluid, thereby removing the bound amyloid beta fibrils from the brain.

### Example 5

### Dual-functioning Magnetic Nanoparticle—Imaging and Removal

A magnetic nanoparticle as described in Example 1 is configured to carry a contrast imaging agent that can be visualized by an MRI. The imaging agent has T1 contrast, T2 contrast, or both contrasting effects simultaneously.

The contrast-magnetic nanoparticle is administered to an Alzheimer's patient. The nanoparticle is able to cross the blood brain barrier and bind to amyloid beta protein fibrils as described in Example 4. The patient undergoes an Mill to image the amyloid beta accumulation in the brain.

A magnetic shunt is positioned into the temporal horns of the patient's brain, and the nanoparticle-amyloid beta complexes are removed as described in Example 4.

A second Mill is performed on the patient to determine the extent of amyloid beta clearance from the brain.

### Example 6

### Removal of Viral Particles from Brain

A patient contracts a human cytomegalovirus (hCMV).

Magnetic nanoparticles are prepared and covered with transferrin and anti-CMV antibodies. These nanoparticles are administered to the patient. Transferrin allows the nanoparticles to cross the blood brain barrier through receptor-mediated transfer. The anti-CMV antibodies allow the nanoparticles to bind to hCMV viral particle.

A magnetic shunt as described in Example 2 is inserted into the brain of the patient. The magnetic field and the opening and closing of the shunt is controls as described in Example 4. The magnetic shunt is able to accumulate and remove nanoparticle-hCMV complexes from the brain.

### Example 7

### Removal of Cholesterol from Blood Vessels

Magnetic nanoparticles are prepared and coated with anti-cholesterol antibodies. The nanoparticles are administered to the blood stream of a patient. The nanoparticles bind to cholesterol plaques in the patient's blood.

A magnetic field generating venous catheter is positioned into a view of the patient. The venous catheter is configured similar to the magnetic field generating shunt described in Example 2. The distal end of the catheter emits a magnetic field that attracts and magnetically couples with the nanoparticle-cholesterol complexes. These complexes are accumulated at the distal tip of the catheter.

The magnetic field is turned off while the catheter is opened, allowing the accumulated nanoparticle-cholesterol complexes to be drawn into the catheter and removed from the patient.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for removing a substance from a tissue, the method comprising:
(a) providing the tissue comprising the substance, a magnetic coupling nanoparticle comprising a tagging element and a magnetic element, wherein the tagging element is capable of binding to the substance, and a magnetic field generating removal device configured to magnetically couple with the magnetic element of the nanoparticle; and
(b) drawing the substance from the tissue using the magnetic field generating removal device when the substance is bound to the nanoparticle, thereby removing the substance from the tissue.

2. The method of claim 1, provided that the tissue is brain tissue.

3. The method of claim 1, provided that the substance comprises a misfolded protein, prion, microorganism, viral particle, pathogen, infectious agent, toxic agent, organic molecule, element, mercury, lead, cancer cells, inflammatory cells, or an associated plurality of the same.

4. The method of claim 1, provided that the tagging element comprises an antibody or peptide that binds to a Tau protein or amyloid beta.

5. The method of claim 1, wherein the magnetic field generating removal device generates a magnetic field that is capable of being turned on and off.

6. The method of claim 5, provided that the substance-bound nanoparticle magnetically couples with the magnetic field generating removal device when the magnetic field is on, thereby accumulating the substance.

7. The method of claim 6, further comprising releasing the substance-bound nanoparticle from the magnetic field generating removal device when the magnetic field is off.

8. The method of claim 1, provided that the magnetic field generating removal device comprises a conduit configured to comprise a first configuration where the conduit is closed, and a second configuration where the conduit is open such that the substance-bound nanoparticle may enter the conduit.

9. The method of claim 1, provided that the magnetic coupling nanoparticle is administered to an individual having the tissue, and the magnetic field removal device is positioned within proximity to the tissue sufficient to draw the substance-bound nanoparticle from the individual using the magnetic field generating removal device.

10. A magnetic field generating device comprising a conduit configured to magnetically couple with and receive a magnetic element when in proximity to the magnetic element.

11. The device of claim 10, provided that generation of the magnetic field is capable of being turned on and off.

12. The device of claim 10, provided that the device comprises a shunt or catheter having a distal end from which the magnetic field is emitted.

13. The device of claim 10, provided that the conduit is configured to comprise a closed configuration and an open configuration, wherein the magnetic field is configured to be off when the conduit is open and the magnetic field is configured to be on when the conduit is closed.

14. The device of claim 10, further comprising a valve.

15. A system comprising:
a. a nanoparticle comprising
i. a tagging element configured to bind the nanoparticle to a substance; and
ii. a magnetic element configured to magnetically couple the nanoparticle to a magnetic field; and
b. a removal device configured to generate the magnetic field.

16. The system of claim 15, provided that the magnetic element comprises ferrous oxide, ferric oxide, ferromagnetic iron oxide, maghemite, gamma-Fe2O3, magnetite, Fe3O4, or a combination thereof.

17. The system of claim 15, provided that the substance is a misfolded protein, prion, microorganism, viral particle, pathogen, infectious agent, toxic agent, organic molecule, element, mercury, lead, cancer cells, inflammatory cells, or an associated plurality of the same.

18. The system of claim 15, provided that the removal device comprises a shunt, catheter, implant, or intermittent device.

19. The system of claim 15, provided that generation of the magnetic field is adjustable, and/or the magnetic field is capable of being turned on and off.

20. The system of claim 15, provided that the removal device comprises a conduit configured to comprise an open configuration and a closed configuration.
